(19) United States
(12) Patent Application Publication
(10) Pub. No.: US 2023/0263690 A1
(43) Pub. Date: Aug. 24, 2023

(54) GAIT ASSIST

(71) Applicant: Timothy Roy Dean, Leesburg, GA (US)

(72) Inventor: Timothy Roy Dean, Leesburg, GA (US)

(21) Appl. No.: 18/113,856

(22) Filed: Feb. 24, 2023

Related U.S. Application Data
(60) Provisional application No. 63/313,525, filed on Feb. 24, 2022.

(51) Int. Cl. A61H 3/00; A61H 1/02

(52) U.S. Cl. CPC A61H 3/00; A61H 1/0262; A61H 2003/007; A61H 2201/1642; A61H 2201/165; A61H 2201/1261

(57) ABSTRACT

Various examples are provided related to gait assist. In one example, a gait assist assembly includes a foot strap that can be positioned on a foot of a lower extremity; an inferior knee strap that can be positioned below a knee of the lower extremity; a superior knee strap that can be positioned above the knee; belt straps or vest; and at least one resistance band or tube for connection to or through a combination of connectors of the foot strap, the inferior knee strap, the superior knee strap, and/or belt straps or vest. In another example, a gait assist assembly includes a foot strap; a knee brace including inferior and superior knee connections; a vest or belt straps; and at least one resistance band or tube for connection to or through a combination of connectors of the foot strap, the knee brace, and/or vest or belt straps.

### CROSS REFERENCE TO RELATED APPLICATIONS

This application claims priority to, and the benefit of, co-pending U.S. provisional application entitled “Gait Assist” having Ser. No. 63/313,525, filed Feb. 24, 2022, which is hereby incorporated by reference in its entirety.

### BACKGROUND

A wide range of injuries or other medical conditions can affect the gait of an individual, which can lead to other physical effect. Rehabilitation is often prescribed to assist in reestablishing a normal gait of the individual.

### SUMMARY

Aspect of the present disclosure are related to gait assistance. In one aspect, among others, a gait assist assembly comprises a foot strap configured for positioning on a foot of a lower extremity; an inferior knee strap configured for positioning below a knee of the lower extremity; a superior knee strap configured for positioning above the knee of the lower extremity; belt straps or vest; and at least one resistance band or tube for connection to or through a combination of connectors of two or more of the foot strap, the inferior knee strap, the superior knee strap, belt straps or vest. The at least one resistance band or tube can pass through connectors of the foot strap, the inferior knee strap and the superior knee strap and can be attached to connectors of the belt straps or vest. The connectors of the belt straps or vest can be located at the front or the back of a user.

In one or more aspects, the at least one resistance band or tube can provide a defined resistance. The belt straps can be attached to a belt, which can be configured to be worn around a waist of an individual. The vest can be configured to be worn around a torso of an individual. The connectors can comprise buckles, D-rings, or a combination thereof. The at least one resistance band or tube can extend over a knee guard between the superior knee strap and the inferior knee strap. The knee guard can be attached to the superior knee strap, the inferior knee strap, or both.

In another aspect, a gait assist assembly comprises a foot strap configured for positioning on a foot of a lower extremity; a knee brace comprising inferior knee connections configured for positioning below a knee of the lower extremity and superior knee connections configured for positioning above the knee of the lower extremity; a vest or belt straps; and at least one resistance band or tube for connection to or through a combination of connectors of two or more of the foot strap, the knee brace, the vest, or belt straps. The at least one resistance band or tube can pass through connectors of the foot strap and the knee brace and can be attached to connectors of the vest or belt straps. The connectors of the belt straps or vest can be located at the front or the back of a user.

In one or more aspects of these embodiments, the at least one resistance band or tube can provide a defined resistance. The belt straps can be attached to a belt configured to be worn around a waist of an individual. The vest can be configured to be worn around a torso of an individual. The connectors can comprise buckles, D-rings, or a combination thereof. The at least one resistance band or tube can extend over the knee brace between the superior knee connections and the inferior knee connections. The knee brace can comprise an inferior knee strap attached to the inferior knee connections and a superior knee strap attached to the superior knee connections.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates phases of a gait pattern, in accordance with various embodiments of the present disclosure.

FIGS. 2A-2C illustrate examples of gait assist apparatus, in accordance with various embodiments of the present disclosure.

FIG. 3 illustrates a selection range for resistance of the gait apparatus of FIGS. 2A-2C, in accordance with various embodiments of the present disclosure.

### DETAILED DESCRIPTION

Disclosed herein are various examples related to methods, systems and apparatus for gait assistance. Reference will now be made in detail to the description of the embodiments as illustrated in the drawings, wherein like reference numbers indicate like parts throughout the several views.

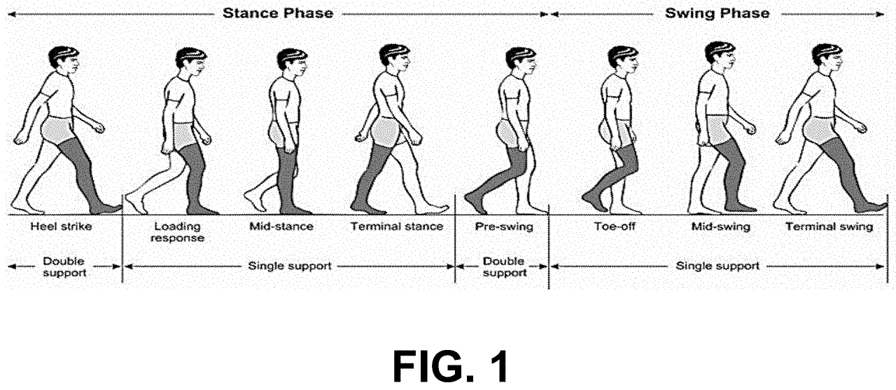

FIG. 1 illustrates an example of the phases of a gait pattern. During the stance phase, the heel of an individual's leg strikes providing double support with both legs. As the individual advances, single support is provided as the leg passes through a loading response, mid-stance, and terminal stance. During this sequence, the other leg is lifted and advanced to establish double support during a pre-swing period. This is followed by a swing phase where the individual's leg passes through a toe-off, mid-swing and terminal swing as it is lifted and advanced to reestablish support. During the swing phase, the other leg provides single support of the individual.

The gait assist was designed to aid in patients with any gait abnormalities limiting a normal gait pattern secondary to medical diagnosis. The gait assist, designed for utilization for ortho, neuro, strengthening, and balance rehab through resistance strengthening. The gait assist can be utilized for the following diagnoses:
CVAs;
Spinal cords;
TBIs;
Orthopedic; and/or
General deconditioned.
   The gait assist can facilitate hip flexion/extension, knee flexion/extension, and/or dorsiflexion to initiate lower extremity movement to reestablish a normal gait pattern. The gait assist can also be utilized to decrease tone through increasing weight bearing through approximation when donned. The gait assist can also strengthen through resistance which can be decreased as the patient gets stronger with increased range of motion (ROM). The patient can achieve concentric and eccentric strengthening.

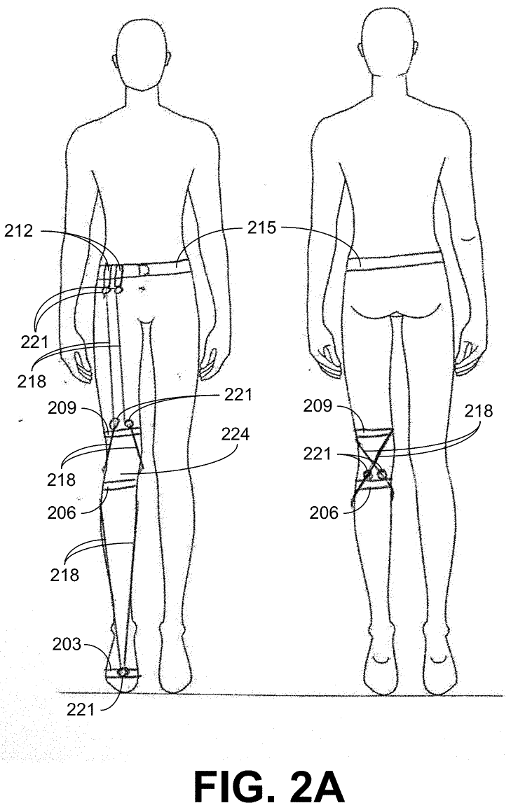

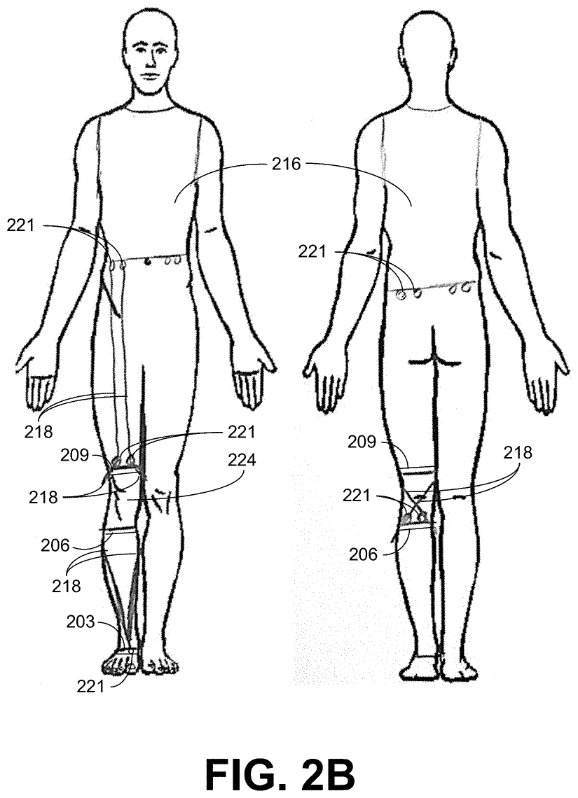

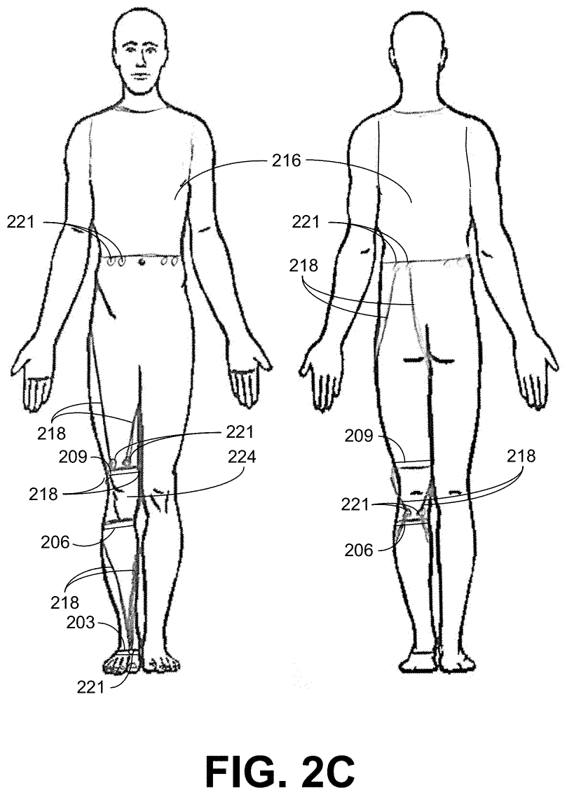

FIG. 2A illustrates an example of a gait assist apparatus. As shown, the gait assist apparatus can comprise a foot strap 203, inferior knee strap 206, superior knee strap 209, and belt straps 212 that can be attached to a belt 215. Resistance bands or tubes 218 (e.g., Thera-Bands®) can be coupled through or between connectors 221 on the straps to provide tension or resistance. For example, resistance bands 218 can be connected to belt straps 212 or foot strap 203 while extending through one or more connectors 221 of one or more of the other straps, or resistance bands 218 can be connected between the foot strap 203 and inferior knee strap 206, between the inferior knee strap 206 and superior knee strap 209, between the superior knee strap 209 and the belt straps 212, and/or between other combinations of the straps. In some cases, the foot strap 203 can be, e.g., a foot glove or sock for foot attachment or a foot clip configured to adhere to different types of shoes.

The connectors 221 can comprise rings (e.g., D-rings), loops, grommets, clips, latches or other appropriate connectors or combination of connectors. In some cases, the connectors can be configured to swivel to allow for movement or rotation of the resistance bands 218 as the leg moves. The gait assist apparatus can also include a knee brace or guard 224 to provide padding between the resistance bands 218 and the knee of the individual. In some embodiments, the knee brace or guard 224 can be integrated with one or both of the inferior knee strap 206 and superior knee strap 209 to form a single unit. Other guards (e.g., knee guard or thigh guards may also be included for protection or padding. In some applications, knee straps can be added for knee brace type applications. In various embodiments, the gait assist apparatus can include weighted vest latches or connectors at a posterior portion of a vest. Resistance bands can be connected to provide or initiate hip extension.

FIG. 2B illustrates another example of the gait assist apparatus utilizing a vest 216 for hip/knee flexion and/or dorsiflexion instead of a belt 215 and belt straps 212. As shown, the gait assist apparatus can comprise a foot strap 203, knee brace or guard 224 with inferior knee strap 206 and superior knee strap 209, and vest 216 with anterior/posterior buckles or connectors for resistance band attachment. Resistance bands or tubes 218 (e.g., Thera-Bands®) can be coupled through or between connectors 221 to provide tension or resistance. For example, resistance bands 218 can be connected to the vest 216 or foot strap 203 while extending through one or more connectors 221 of one or more of the other straps, or resistance bands 218 can be connected between the foot strap 203 and inferior knee strap 206 of knee guard 224, between the inferior knee strap 206 and superior knee strap 209, between the superior knee strap 209 of knee guard 224 and the vest connectors 221, and/or between other combinations of the straps/connectors. In some cases, the foot strap 203 can be, e.g., a foot glove or sock for foot attachment or a foot clip configured to adhere to different types of shoes.

FIG. 2C illustrates another example of the gait assist apparatus utilizing the vest 216 for hip/knee extension. This arrangement can also be used with the belt 215 and belt straps 212 of FIG. 2A. As shown, the gait assist apparatus can comprise the foot strap 203, knee brace or guard 224 with inferior knee strap 206 and superior knee strap 209, and vest 216 with anterior/posterior buckles or connectors for resistance band attachment. Resistance bands or tubes 218 (e.g., Thera-Bands®) can be coupled through or between connectors 221 to provide tension or resistance. For example, resistance bands 218 can be connected to the vest 216 or foot strap 203 while extending through one or more connectors 221 of one or more of the other straps, or resistance bands 218 can be connected between the foot strap 203 and inferior knee strap 206 of knee guard 224, between the inferior knee strap 206 and superior knee strap 209, between the superior knee strap 209 of knee guard 224 and the vest connectors 221, and/or between other combinations of the straps/connectors. In some cases, the foot strap 203 can be, e.g., a foot glove or sock for foot attachment or a foot clip configured to adhere to different types of shoes.

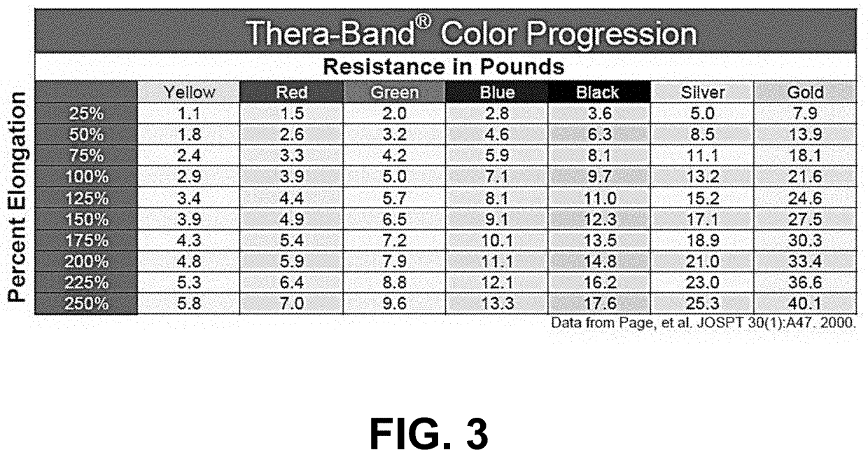

FIG. 3 is a table illustrating a range of Thera-Band® resistance bands 218 that can be used with the gait apparatus of FIGS. 2A-2C. Different strength/color bands can be selected depending on the task and individual specifics. The resistance bands or tubes 218 can be varied over time as the therapy progresses. Using the resistance bands 218, the gait assist apparatus can:
Strengthen the leg or lower extremity through the resistance bands;
Facilitate hip, knee flexion, and dorsiflexion (simulates NDT);
Facilitate hip and knee extension;
Aid in faster return of normal gait pattern (heel to toe);
Increase erect posture;
Increase motor control;
Facilitate concentric and/or eccentric contractions;
Increase approximation in all lower extremity joints; and/or
Facilitate nerve regeneration secondary to joint approximation.

While the example of FIG. 2A shows combinations of two resistance bands 218 connected between the straps, other combinations of resistance bands 218 can be used as illustrated in FIGS. 2B and 2C. For example, additional bands can be included to increase the resistance. Different color resistance bands can be utilized with specific points on the band for the individual's height.

Application of the gait assist apparatus of FIG. 2A will now be described. Initially, belt straps 212 can be applied on an appropriate side of the belt 215, the superior knee strap 209 can be positioned at the appropriate location of the lower extremity (e.g., above the knee), the inferior knee strap 206 can be positioned at the appropriate location of the lower extremity (e.g., below the knee), and the foot strap 203 on the foot of the lower extremity. Resistance straps 218 can then be connected between the straps using the connectors 221. The resistance straps 218 can be sized based upon the prescribed resistance, length between straps, etc. A knee guard 224 (or other guard) can be applied before or after the resistance bands 218 are connected to the straps 203, 206, 209, 212.

A first example for donning the gait assist apparatus using the Thera-Bands® resistance bands can include:
Step 1: Don the gait belt straps 212 (e.g., two) on gait belt in the positioned on desired side of the lower extremity.
Step 2: Don the superior knee strap 209 on the lower extremity, e.g., about 2″ above center of kneecap with connectors (e.g., D-rings) 221 on an anterior side of lower extremity facing up.
Step 3: Don the inferior knee strap 206 on the lower extremity, e.g., about 3″ below center of kneecap with the connectors (e.g., D-rings) 221 on a posterior side of lower extremity facing up.
Step 4: Don the foot strap 203 on the lower extremity, e.g., about 1.5″ from tip of toes with the connector (e.g., D-ring) 221 facing up.
Step 5: Choose one or more appropriate resistance band (e.g., the desired colored Thera-Band® resistance band) 218 for the individual's deficit.
Step 6: Start at the foot strap 203, divide the resistance band 218 in half, once the resistance band 218 is through the foot connector (e.g., D-ring) 221, run the medial portion of the resistance band 218 through the medial connector (e.g., D-ring) 221 of the inferior knee strap 206 and the lateral connector (e.g., D-ring) 221 of the superior knee strap 209 followed by attaching to lateral gait belt connectors (e.g., D-rings) 221 with desired resistance. Next run the lateral portion of the resistance band 218 through the lateral connector (e.g., D-ring) 221 of the inferior knee strap 206 and the medial portion of the superior knee strap 209, then attach to medial gait belt connector (e.g., D-ring) 221 with desired resistance.

A second example for donning the gait assist apparatus using the Thera-Bands® resistance bands can include:
Step 1: Don a weighted vest comprising connectors (e.g., D-rings) 221.
Step 2: Don superior knee strap 209 on the lower extremity, e.g., about 2″ above center of kneecap with the connectors (e.g., D-rings) 221 on an anterior side of lower extremity facing up.
Step 3: Don inferior knee strap 206 on the lower extremity, e.g., about 3″ below center of kneecap with the connectors (e.g., D-rings) 221 on a posterior side of lower extremity facing up.
Step 4: Don the foot strap 203 on the lower extremity, e.g., about 1.5″ from tip of toes with connector (e.g., D-ring) 221 facing up.
Step 5: Choose appropriate one or more resistance band (e.g., the desired colored Thera-Band® resistance band) 218 for the individual's deficit.
Step 6: Start at the foot strap 203, divide the resistance band 218 in half, once the resistance band 218 is through the foot connector (e.g., D-ring) 221, run the medial portion of the resistance band 218 through the medial connector (e.g., D-ring) 221 of the inferior knee strap 206 and the lateral connector (e.g., D-ring) 221 of the superior knee strap 209 followed by attaching to lateral vest latch with desired resistance/height. Next run the lateral portion of the resistance band 218 through the lateral connector (e.g., D-ring) 221 of the inferior knee strap 206 and the medial portion of the superior knee strap 209, then attach to medial vest latch with desired resistance/height.

Application of the gait assist apparatus of FIGS. 2B and 2C will now be described. Initially, the vest 216 can be applied, the knee brace can be positioned at the appropriate lower extremity with the superior knee connections facing up and the foot strap 203 on the foot of the lower extremity. Resistance straps 218 can then be connected between the straps using the connectors 221. The resistance straps 218 can be sized based upon the prescribed resistance, length between straps, etc.

An example for donning the gait assist apparatus with a vest 216 as shown in FIGS. 3B and 3C using the Thera-Bands® resistance bands can include:
Step 1: Don the vest 216.
Step 2: Don the knee brace 224 on the lower extremity.
Step 3: Don the foot strap 203 on the lower extremity, e.g., about 1.5″ from tip of toes with the connector (e.g., D-ring) 221 facing up.
Step 4: Choose one or more appropriate resistance band (e.g., the desired colored Thera-Band® resistance band) 218 for the individual's deficit.
Step 5: A first example for donning the gait assist apparatus using the Thera-Bands® resistance bands can include:
Step 1: Don the vest
Step 2: Don the knee brace 209 on the lower extremity
Step 3: Don the foot strap 203 on the lower extremity, e.g., about 1.5″ from tip of toes with the connector (e.g., D-ring) 221 facing up.
Step 4: Choose appropriate resistance band (e.g., the desired colored Thera-Band® resistance band) 218 for the individual's deficit.
Step 5: Start at the foot strap 203, divide the resistance band 218 in half, once the resistance band 218 is through the foot connector (e.g., D-ring) 221, run the medial portion of the resistance band 218 through the inferior connector (e.g., D-ring) 221 of the knee brace 224 and the superior connector (e.g., D-ring) 221 of the knee brace 224 followed by attaching to lateral vest connectors (e.g., D-rings or buckles) 221 with desired resistance. Next run the lateral portion of the resistance band 218 through the inferior connector (e.g., D-ring) 221 of the knee brace 224 and the medial portion of the knee brace 209, then attach to medial vest buckles (e.g., D-ring) 221 with desired resistance.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A gait assist assembly, comprising:
a foot strap configured for positioning on a foot of a lower extremity;
an inferior knee strap configured for positioning below a knee of the lower extremity;
a superior knee strap configured for positioning above the knee of the lower extremity;
belt straps or vest; and
at least one resistance band or tube for connection to or through a combination of connectors of two or more of the foot strap, the inferior knee strap, the superior knee strap, belt straps or vest.

2. The gait assist assembly of claim 1, wherein the at least one resistance band or tube passes through connectors of the foot strap, the inferior knee strap and the superior knee strap and is attached to connectors of the belt straps or vest.

3. The gait assist assembly of claim 1, wherein the at least one resistance band or tube provides a defined resistance.

4. The gait assist assembly of claim 1, wherein the belt straps are attached to a belt.

5. The gait assist assembly of claim 4, wherein the belt is configured to be worn around a waist of an individual.

6. The gait assist assembly of claim 1, wherein the vest is configured to be worn around a torso of an individual.

7. The gait assist assembly of claim 6, wherein connectors on the vest comprise buckles.

8. The gait assist assembly of claim 1, wherein the connectors comprise D-rings.

9. The gait assist assembly of claim 1, wherein the at least one resistance band or tube extends over a knee guard between the superior knee strap and the inferior knee strap.

10. The gait assist assembly of claim 9, wherein the knee guard is attached to the superior knee strap, the inferior knee strap, or both.

11. A gait assist assembly, comprising:
a foot strap configured for positioning on a foot of a lower extremity;
a knee brace comprising inferior knee connections configured for positioning below a knee of the lower extremity and superior knee connections configured for positioning above the knee of the lower extremity;
a vest or belt straps; and
at least one resistance band or tube for connection to or through a combination of connectors of two or more of the foot strap, the knee brace, the vest, or belt straps.

12. The gait assist assembly of claim 11, wherein the at least one resistance band or tube passes through connectors of the foot strap and the knee brace and is attached to connectors of the vest or belt straps.

13. The gait assist assembly of claim 11, wherein the at least one resistance band or tube provides a defined resistance.

14. The gait assist assembly of claim 11, wherein the belt straps are attached to a belt configured to be worn around a waist of an individual.

15. The gait assist assembly of claim 11, wherein the vest is configured to be worn around a torso of an individual.

16. The gait assist assembly of claim 15, wherein connectors on the vest comprise buckles.

17. The gait assist assembly of claim 11, wherein the connectors comprise D-rings.

18. The gait assist assembly of claim 11, wherein the at least one resistance band or tube extends over the knee brace between the superior knee connections and the inferior knee connections.

19. The gait assist assembly of claim 11, wherein the knee brace comprises an inferior knee strap attached to the inferior knee connections and a superior knee strap attached to the superior knee connections.
